(19) United States
(12) Patent Application Publication
(10) Pub. No.: US 2012/0004298 A1
(43) Pub. Date: Jan. 5, 2012

(54) OPHTHALMIC DEVICES CONTAINING CHEMOKINE ANTAGONISTS

(76) Inventors: Hassan Chaouk, Jacksonville, FL (US); Dijana Draganovic, Jacksonville, FL (US); Vandeeta Khanolkar, Jacksonville, FL (US)

(21) Appl. No.: 13/172,269

(22) Filed: Jun. 29, 2011

Related U.S. Application Data
(60) Provisional application No. 61/359,963, filed on Jun. 30, 2010.

(51) Int. Cl. A61K 31/351; A61P 29/00; A61P 27/02; C07D 309/14

(52) U.S. Cl. 514/459; 549/424

(57) ABSTRACT

Ionic ophthalmic devices, methods of treating chemoattractant cytokine receptor 2 (CCR2) mediated inflammatory conditions, and methods of making such devices are disclosed herein.

### RELATED DOCUMENTS

This application claims priority to U.S. Provisional Pat. App. Ser. No. 61/359,963, which was filed on Jun. 30, 2010,

### FIELD OF THE INVENTION

This invention related to devices containing antagonists to chemoattractant cytokine receptor 2 (CCR2) and methods of making the same.

### BACKGROUND

Chemoattractant cyctokine receptor 2 (CCR2) is play a role in inflammatory disease states. In animal models antagonists to this receptor suppress inflammatory responses in allergic conditions. There are small molecule antagonists of this receptor, known a pheynoamino substituted quaternary salts. See U.S. Pat. Pub. No. 2006/0293379, which is hereby incorporated by reference in its entirety. Those antagonists are potentially useful in treating inflammatory diseases of the eye, including but not limited to uveitis, inflammation after surgery, allergic conjunctivitis, dry eye, allergic rhinitis, and the like. It would be useful to deliver CCR2 antagonists directly to the eye using an ophthalmic device. Incorporating such small molecules into an ophthalmic lens, so that enough drug is absorbed would be useful and this invention meeting that need.

### BRIEF DESCRIPTION OF THE DRAWING

FIG. 1 illustrates the release of a compound of Formula A from an ionic ophthalmic device.

### DETAILED DESCRIPTION OF THE INVENTION

This invention includes an ionic ophthalmic device comprising an effective amount of a phenylamino substituted quaternary salt. As used herein “phenylamino substituted quaternary salt” refers to chemical substances of Formula (I) that are disclosed in U.S. Pat. Pub. No. 2006/0293379, as well as mixtures thereof.

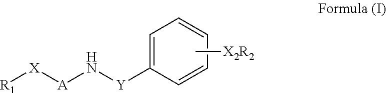

and pharmaceutically acceptable forms thereof, wherein
A is carbonyl, thiocarbonyl or sulfonyl;
X is a bond or —CH═CH—;
R₁ is selected from
   aryl optionally substituted by one or more lower alkyl, —(CH₂)ₙ—CF₃, lower alkoxy, alkoxycarbonyl, cyano, halogen or phenyl optionally substituted by lower alkyl, —(CH₂)ₙ—CF₃, lower alkoxy, alkoxycarbonyl, cyano or halogen;
   C₅-C₁₅ cycloalkyl optionally substituted by one or more lower alkyl, —(CH₂)ₙ—CF₃, lower alkoxy, aryl, halogen-substituted aryl, alkoxycarbonyl, cyano or halogen; or,
   heterocyclyl optionally substituted by one or more lower alkyl, —(CH₂)ₙ—CF₃, lower alkoxy, aryl, aryl-lower alkyl, halogen-substituted aryl, alkoxycarbonyl, cyano or halogen;
n is 0, 1, 2, 3 or 4;
Y is a bond or —CH₂—;
X₂ is —(CH₂)ₘ— wherein m is 1 or 2;
R₂ is —N⁺(R₄R₅)—ZR₃;
Z is —(CH₂)ₚ— wherein p is 0, 1 or 2;
R₃ is selected from
   aryl optionally substituted with one or more lower alkyl, —(CH₂)ₙ—CF₃, lower alkoxy, aryl, halogen-substituted aryl, alkoxycarbonyl, cyano or halogen;
   C₅-C₁₅ cycloalkyl optionally substituted with one or more lower alkyl, —(CH₂)ₙ—CF₃, lower alkoxy, aryl, halogen-substituted aryl, alkoxycarbonyl, cyano or halogen; or,
   heterocyclyl optionally substituted with one or more lower alkyl, —(CH₂)ₙ—CF₃, lower alkoxy, aryl, halogen-substituted aryl, alkoxycarbonyl, cyano or halogen; wherein, when heterocyclyl is attached via a carbon atom ring member and a heteroatom ring member is adjacent to said carbon atom, then p is 1 or 2;
R₄ and R₅ are each individually lower alkyl or lower alkenyl;
alternatively, R₄ and R₅ combine with the nitrogen atom of Formula (I) to form a heterocyclyl ring of 5 to 9 total ring atoms optionally containing one of an oxygen or sulfur ring atom, wherein the heterocyclyl ring nitrogen atom is substituted with one of lower alkyl or lower alkenyl to form a quaternary salt, and wherein —ZR₃ is absent and the heterocyclyl ring is optionally substituted with aryl optionally substituted with one or more lower alkyl, —(CH₂)ₙ—CF₃, lower alkoxy, aryl, halogen-substituted aryl, alkoxycarbonyl, cyano or halogen.
   Preferred pheynylamio substituted quaternary salts are selected from the group consisting of compounds of Table 1.

TABLE 1

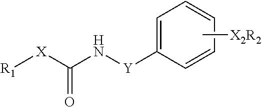

or a pharmaceutically acceptable form thereof, wherein R₁, X, Y and X₂R₂ are
dependently selected from

| Cpd | R₁ | X | Y | X₂R₂ |
|---|---|---|---|---|
| 1 | 3-Br-phenyl | —CH═CH— | —CH₂— | 4-CH₂—N⁺(CH₃)₂-cyclohexyl, |
| 2 | 3-Br-phenyl | bond | —CH₂— | 4-CH₂—N⁺(CH₃)₂-cyclohexyl, |
| 3 | 3-CF₃-phenyl | bond | —CH₂— | 4-CH₂—N⁺(CH₃)₂-cyclohexyl, |
| 4 | 3,4-Cl₂-phenyl | —CH═CH— | —CH₂— | 4-CH₂—N⁺(CH₃)₂-tetrahydro-pyran-4-yl, |
| 5 | 3-Br-phenyl | —CH═CH— | —CH₂— | 4-CH₂—N⁺(CH₃)₂-tetrahydro-pyran-4-yl, |
| 6 | phenyl | bond | bond | 4-CH₂—N⁺(CH₃)₂-tetrahydro-pyran-4-yl, |
| 7 | 3,4-Cl₂-phenyl | bond | bond | 3-CH₂—N⁺(CH₃)₂-tetrahydro-pyran-4-yl, |
| 8 | 3-Br-phenyl | bond | bond | 3-CH₂—N⁺(CH₃)₂-tetrahydro-pyran-4-yl, |
| 9 | 2,3-Cl₂-phenyl | bond | bond | 4-CH₂—N⁺(CH₃)₂-tetrahydro-pyran-4-yl, |
| 10 | 2,4-Cl₂-phenyl | bond | bond | 4-CH₂—N⁺(CH₃)₂-tetrahydro-pyran-4-yl, |
| 11 | 2,5-Cl₂-phenyl | bond | bond | 4-CH₂—N⁺(CH₃)₂-tetrahydro-pyran-4-yl, |
| 12 | 2,6-Cl₂-phenyl | bond | bond | 4-CH₂—N⁺(CH₃)₂-tetrahydro-pyran-4-yl, |
| 13 | 2-Cl-phenyl | bond | bond | 4-CH₂—N⁺(CH₃)₂-tetrahydro-pyran-4-yl, |
| 14 | 3,4-Cl₂-phenyl | bond | bond | 4-CH₂—N⁺(CH₃)₂-bicyclo[2.2.1]hept-2-yl, |
| 15 | 3,4-Cl₂-phenyl | bond | bond | 4-CH₂—N⁺(CH₃)₂-(2S)-CH₂-tetrahydro- |
| | | | | furan-2-yl, |
| 16 | 3,4-Cl₂-phenyl | bond | bond | 4-CH₂—N⁺(CH₃)₂-(2R)-CH₂-tetrahydro- |
| | | | | furan-2-yl, |
| 17 | 3,4-Cl₂-phenyl | bond | bond | 4-CH₂—N⁺(CH₃)₂-tetrahydro-pyran-4-yl, |
| 18 | 3,4-Cl₂-phenyl | bond | bond | 4-CH₂—N⁺(CH₃)₂-CH₂-tetrahydro-pyran- |
| | | | | 4-yl, |
| 19 | 3,4-Cl₂-phenyl | bond | bond | 4-CH₂—N⁺(CH₃)₂-tetrahydro-thien-3-yl, |
| 20 | 3,4-Cl₂-phenyl | bond | bond | 4-CH₂—N⁺(CH₃)₂-tetrahydro-thiopyran-4- |
| | | | | yl, |
| 21 | 3,4-Cl₂-phenyl | bond | bond | 4-CH₂—N⁺[(CH₃)(CH₂CH₃)]-tetrahydro- |
| | | | | pyran-4-yl, |
| 22 | 3,4-Cl₂-phenyl | bond | bond | 4-CH₂—N⁺{(CH₃)[(CH₂)₂CH₃)]}- |
| | | | | tetrahydro-pyran-4-yl, |
| 23 | 3,5-Cl₂-phenyl | bond | bond | 4-CH₂—N⁺(CH₃)₂-tetrahydro-pyran-4-yl, |
| 24 | 3-Br-phenyl | bond | bond | 4-CH₂—N⁺(CH₃)₂-tetrahydro-pyran-4-yl, |
| 25 | 2-CH₃-3-Cl₂-phenyl | bond | bond | 4-CH₂—N⁺(CH₃)₂-tetrahydro-pyran-4-yl, |
| 26 | 3-Cl-4-F-phenyl | bond | bond | 4-CH₂—N⁺(CH₃)₂-tetrahydro-pyran-4-yl, |
| 27 | 3-Cl-4-OCH₃-phenyl | bond | bond | 4-CH₂—N⁺(CH₃)₂-tetrahydro-pyran-4-yl, |
| 28 | 3-Cl-4-CH₃-phenyl | bond | bond | 4-CH₂—N⁺(CH₃)₂-tetrahydro-pyran-4-yl, |
| 29 | 3-Cl-phenyl | bond | bond | 4-CH₂—N⁺(CH₃)₂-tetrahydro-pyran-4-yl, |
| 30 | 3-CN-phenyl | bond | bond | 4-CH₂—N⁺(CH₃)₂-tetrahydro-pyran-4-yl, |
| 31 | 3-OCH₃-phenyl | bond | bond | 4-CH₂—N⁺(CH₃)₂-tetrahydro-pyran-4-yl, |
| 32 | 2-CH₃-4-Cl-phenyl | bond | bond | 4-CH₂—N⁺(CH₃)₂-tetrahydro-pyran-4-yl, |
| 33 | 3-CF₃-4-Cl-phenyl | bond | bond | 4-CH₂—N⁺(CH₃)₂-tetrahydro-pyran-4-yl, |
| 34 | 4-Cl-phenyl | bond | bond | 4-CH₂—N⁺(CH₃)₂-tetrahydro-pyran-4-yl, |
| 35 | 2-CH₃-5-Cl-phenyl | bond | bond | 4-CH₂—N⁺(CH₃)₂-tetrahydro-pyran-4-yl, |
| 36 | 3,4-Cl₂-phenyl | bond | bond | 4-(CH₂)₂—N⁺(CH₃)₂-tetrahydro-pyran-4- |
| | | | | yl, |
| 37 | 3-Br-phenyl | bond | bond | 4-(CH₂)₂—N⁺(CH₃)₂-tetrahydro-pyran-4- |
| | | | | yl, |
| 38 | 3-Br-phenyl | —CH═CH— | bond | 3-CH₂—N⁺(CH₃)₂-tetrahydro-pyran-4-yl, |
| 39 | 3,4-Cl₂-phenyl | bond | —CH₂— | 4-CH₂—N⁺(CH₃)₂-tetrahydro-pyran-4-yl, |
| 40 | 3,4-Cl₂-phenyl | —CH═CH— | bond | 4-CH₂—N⁺(CH₃)₂-tetrahydro-pyran-4-yl, |
| 41 | 3,4-Cl₂-phenyl | —CH═CH— | bond | 4-CH₂—N⁺(CH₃)₂-tetrahydro-thiopyran-4- |
| | | | | yl, |
| 42 | 3,5-F₂-phenyl | —CH═CH— | bond | 4-CH₂—N⁺(CH₃)₂-tetrahydro-pyran-4-yl, |
| 43 | 3-Br-phenyl | —CH═CH— | bond | 4-CH₂—N⁺(CH₃)₂-tetrahydro-pyran-4-yl, |
| 44 | 3-Br-phenyl | —CH═CH— | bond | 4-CH₂—N⁺(CH₃)₂-tetrahydro-thiopyran-4- |
| | | | | yl, |
| 45 | 3-Cl-phenyl | —CH═CH— | bond | 4-CH₂—N⁺(CH₃)₂-tetrahydro-pyran-4-yl, |
| 46 | 3-F-phenyl | —CH═CH— | bond | 4-CH₂—N⁺(CH₃)₂-tetrahydro-pyran-4-yl, |
| 47 | 4-Br-phenyl | —CH═CH— | bond | 4-CH₂—N⁺(CH₃)₂-tetrahydro-pyran-4-yl, |
| 48 | 3,4-Cl₂-phenyl | —CH═CH— | —CH₂— | 4-CH₂-(1-CH₃-piperidinium), |
| 49 | 3-Br-phenyl | —CH═CH— | —CH₂— | 4-CH₂-(1-CH₃-piperidinium), |
| 50 | 3,4-Cl₂-phenyl | bond | bond | 4-CH₂-(1-CH₃-piperidinium), |
| 51 | 3,4-Cl₂-phenyl | bond | bond | 4-CH₂-(1-CH₃-pyrrolidinium), |
| 52 | 3-Br-phenyl | —CH═CH— | bond | 3-CH₂-(1-CH₃-piperidinium), |
| 53 | 3,4-Cl₂-phenyl | —CH═CH— | bond | 4-CH₂-(1-CH₃-piperidinium), |
| 54 | 3,4-Cl₂-phenyl | —CH═CH— | bond | 4-CH₂-[4-(2-OCH₃-phenyl)-1-CH₃- |
| | | | | piperazin-1-ium], |
| 55 | 3-Br-phenyl | —CH═CH— | bond | 4-CH₂-(1-CH₃-piperidinium), |
| 56 | 3-CF₃-phenyl | bond | bond | 3-CH₂-(1-CH₃-piperidinium), |
| 57 | 3-CF₃-phenyl | —CH═CH— | bond | 4-CH₂-(1-CH₃-piperidinium), |
| 58 | 3,4-Cl₂-phenyl | —CH═CH— | —CH₂— | 4-CH₂-(4-CH₃-morpholin-4-ium), |
| 59 | 3,4-Cl₂-phenyl | bond | bond | 4-CH₂-(4-CH₃-morpholin-4-ium), |
| 60 | 3,4-Cl₂-phenyl | —CH═CH— | bond | 4-CH₂-(4-CH₃-morpholin-4-ium), |
| 61 | 3-Br-phenyl | —CH═CH— | bond | 4-CH₂-(4-CH₃-morpholin-4-ium), |
| 62 | 3-CF₃-phenyl | —CH═CH— | —CH₂— | 4-CH₂-(4-CH₃-morpholin-4-ium), |
| 63 | 3-Br-phenyl | —CH═CH— | bond | 4-CH₂—N⁺[(CH₃)(CH₂CH═CH₂)]- |
| | | | | tetrahydro-thiopyran-4-yl, |
| 64 | 3-CF₃-phenyl | —CH═CH— | —CH₂— | 4-CH₂—N⁺(CH₃)₂-tetrahydro-pyran-4-yl, |
| 65 | 3-CF₃-phenyl | bond | bond | 3-CH₂—N⁺(CH₃)₂-tetrahydro-pyran-4-yl, |
| 66 | 3-CH₃-phenyl | —CH═CH— | bond | 4-CH₂—N⁺(CH₃)₂-tetrahydro-pyran-4-yl, |
| 67 | 3-CF₃-phenyl | bond | bond | 4-CH₂—N⁺(CH₃)₂-tetrahydro-pyran-4-yl, |
| 68 | 3-CF₃-phenyl | —CH═CH— | bond | 4-CH₂—N⁺(CH₃)₂-tetrahydro-pyran-4-yl, |
| 69 | 3-CH₃-phenyl | bond | bond | 4-CH₂—N⁺(CH₃)₂-tetrahydro-pyran-4-yl, |
| 70 | 3,4-Cl₂-phenyl | bond | bond | 4-CH₂—N⁺(CH₃)₂-cycloheptyl, |
| 71 | 3,4-Cl₂-phenyl | —CH═CH— | bond | 4-CH₂—N⁺(CH₃)₂-cyclohexyl, |
| 72 | 3-Br-phenyl | —CH═CH— | bond | 4-CH₂—N⁺(CH₃)₂-cyclohexyl, |
| 73 | 3-Br-phenyl | bond | bond | 4-CH₂—N⁺(CH₃)₂-cyclohexyl, |
| 74 | 3-CF₃-phenyl | bond | bond | 4-CH₂—N⁺(CH₃)₂-cyclohexyl, |
| 75 | 3,4-Cl₂-phenyl | bond | bond | 4-CH₂—N⁺(CH₃)₂-cyclohexyl, |
| 76 | 3-Cl-4-F-phenyl | bond | bond | 4-CH₂—N⁺(CH₃)₂-cyclohexyl, |
| 77 | 2,3-Cl₂-phenyl | bond | bond | 4-CH₂—N⁺(CH₃)₂-cyclohexyl, |
| 78 | 2,6-Cl₂-phenyl | bond | bond | 4-CH₂—N⁺(CH₃)₂-cyclohexyl, |
| 79 | 3-Cl-4-OCH₃-phenyl | bond | bond | 4-CH₂—N⁺(CH₃)₂-cyclohexyl, |
| 80 | 3-Cl-4-CH₃-phenyl | bond | bond | 4-CH₂—N⁺(CH₃)₂-cyclohexyl, |
| 81 | 2,5-Cl₂-phenyl | bond | bond | 4-CH₂—N⁺(CH₃)₂-cyclohexyl, |
| 82 | 3,4-Cl₂-phenyl | bond | bond | 4-CH₂—N⁺(CH₃)₂-cyclopentyl, |
| 83 | 3,4-Cl₂-phenyl | —CH═CH— | bond | 3-CH₂—N⁺(CH₃)₂-cyclohexyl, |
| 84 | 4-F-phenyl | —CH═CH— | bond | 3-CH₂—N⁺(CH₃)₂-cyclohexyl, |
| 85 | 3-(4-CF₃-phenyl)- | bond | bond | 4-CH₂—N⁺(CH₃)₂-tetrahydro-pyran-4-yl, |
| | phenyl | | | |
| 86 | 3-(4-CH₃-phenyl)- | bond | bond | 4-CH₂—N⁺(CH₃)₂-cyclohexyl, |
| | phenyl | | | |
| 87 | 3-(4-CH₃-phenyl)- | bond | bond | 4-CH₂—N⁺(CH₃)₂-tetrahydro-pyran-4-yl, |
| | phenyl | | | |
| 88 | 4-biphenyl | bond | bond | 4-CH₂—N⁺(CH₃)₂-tetrahydro-pyran-4-yl, |
| 89 | 1-naphthalene | bond | bond | 4-CH₂—N⁺(CH₃)₂-tetrahydro-pyran-4-yl, |
| 90 | 2-naphthalene | bond | bond | 4-CH₂—N⁺(CH₃)₂-tetrahydro-pyran-4-yl, |
| 91 | 2-naphthalene | bond | bond | 4-CH₂—N⁺[(CH₃)(CH₂CH₃)]-tetrahydro- |
| | | | | pyran-4-yl, |
| 92 | 2-naphthalene | bond | bond | 4-CH₂—N⁺{(CH₃)[(CH₂)₂CH₃)]}- |
| | | | | tetrahydro-pyran-4-yl, |
| 93 | 7-Br-naphthalen-2-yl | bond | bond | 4-CH₂—N⁺(CH₃)₂-tetrahydro-pyran-4-yl, |
| 94 | 7-Br-naphthalen-2-yl | bond | bond | 4-CH₂—N⁺(CH₃)₂-cyclohexyl, |
| 95 | 6-Br-2H-chromen-3- | bond | bond | 4-CH₂—N⁺(CH₃)₂-tetrahydro-pyran-4-yl, |
| | yl | | | |
| 96 | 6-Cl-2H-chromen-3- | bond | bond | 4-CH₂—N⁺(CH₃)₂-tetrahydro-pyran-4-yl, |
| | yl | | | |
| 97 | 6-Br-2H-chromen-3- | bond | bond | 4-CH₂—N⁺(CH₃)₂-cyclohexyl, |
| | yl | | | |
| 98 | 6-Cl-2H-chromen-3- | bond | bond | 4-CH₂—N⁺(CH₃)₂-cyclohexyl, |
| | yl | | | |
| 99 | 6-Br-2H-chromen-3- | bond | —CH₂— | 4-CH₂—N⁺(CH₃)₂-tetrahydro-pyran-4-yl, |
| | yl | | | |
| 100 | 5,7-Cl₂-2H-chromen- | bond | bond | 4-CH₂—N⁺(CH₃)₂-tetrahydro-pyran-4-yl, |
| | 3-yl | | | |
| 101 | 5,7-Cl₂-2H-chromen- | bond | bond | 4-CH₂—N⁺(CH₃)₂-cyclohexyl, |
| | 3-yl | | | |
| 102 | 6,8-Cl₂-2H-chromen- | bond | bond | 4-CH₂—N⁺(CH₃)₂-tetrahydro-pyran-4-yl, |
| | 3-yl | | | |
| 103 | 6-CH₃-2H-chromen- | bond | bond | 4-CH₂—N⁺(CH₃)₂-tetrahydro-pyran-4-yl, |
| | 3-yl | | | |
| 104 | 6-OCH₃-2H- | bond | bond | 4-CH₂—N⁺(CH₃)₂-tetrahydro-pyran-4-yl, |
| | chromen-3-yl | | | |
| 105 | 6-CH₃-2H-chromen- | bond | bond | 4-CH₂—N⁺(CH₃)₂-cyclohexyl, |
| | 3-yl | | | |
| 106 | 6-OCH₃-2H- | bond | bond | 4-CH₂—N⁺(CH₃)₂-cyclohexyl, |
| | chromen-3-yl | | | |
| 107 | 6,8-Cl₂-2H-chromen- | bond | bond | 4-CH₂—N⁺(CH₃)₂-cyclohexyl, |
| | 3-yl | | | |
| 108 | 6-Cl-2H-chromen-3- | bond | bond | 4-CH₂—N⁺(CH₃)₂-(2R)-CH₂-tetrahydro- |
| | yl | | | furan-2-yl, |
| 109 | 6-Cl-2H-chromen-3- | bond | bond | 4-CH₂—N⁺(CH₃)₂-(2S)-CH₂-tetrahydro- |
| | yl | | | furan-2-yl, |
| 110 | 6-Cl-2H-chromen-3- | bond | bond | 4-CH₂—N⁺(CH₃)₂-(2S)-bicyclo[2.2.1]hept- |
| | yl | | | 2-yl, |
| 111 | 6,8-Cl₂-2H-chromen- | bond | bond | 4-CH₂—N⁺(CH₃)₂-bicyclo[2.2.1]hept-2-yl, |
| | 3-yl | | | |
| 112 | 8-CH₃-2H-chromen- | bond | bond | 4-CH₂—N⁺(CH₃)₂-tetrahydro-pyran-4-yl, |
| | 3-yl | | | |
| 113 | 8-CH₃-2H-chromen- | bond | bond | 4-CH₂—N⁺(CH₃)₂-cyclohexyl, |
| | 3-yl | | | |
| 114 | 6-Cl-8-CH₃-2H- | bond | bond | 4-CH₂—N⁺(CH₃)₂-cyclohexyl, |
| | chromen-3-yl | | | |
| 115 | 6-Cl-8-CH₃-2H- | bond | bond | 4-CH₂—N⁺(CH₃)₂-tetrahydro-pyran-4-yl, |
| | chromen-3-yl | | | |
| 116 | 7,8-Cl₂-2H-chromen- | bond | bond | 4-CH₂—N⁺(CH₃)₂-cyclohexyl, |
| | 3-yl | | | |
| 117 | 6-Cl-8-CH₃-2H- | bond | bond | 4-CH₂—N⁺(CH₃)₂-bicyclo[2.2.1]hept-2-yl, |
| | chromen-3-yl | | | |
| 118 | 6-Cl-8-CH₃-2H- | bond | bond | 4-CH₂—N⁺(CH₃)₂-cycloheptyl, |
| | chromen-3-yl | | | |
| 119 | 6-Cl-8-CH₃-2H- | bond | bond | 4-CH₂—N⁺(CH₃)₂-cyclopentyl, |
| | chromen-3-yl | | | |
| 120 | 6-Cl-8-CH₃-2H- | bond | bond | 4-CH₂—N⁺(CH₃)₂-thien-3-yl, |
| | chromen-3-yl | | | |
| 121 | 6-Cl-8-CH₃-2H- | bond | —CH₂— | 4-CH₂—N⁺(CH₃)₂-tetrahydro-pyran-4-yl, |
| | chromen-3-yl | | | |
| 122 | 6,8-Cl₂-2H-chromen- | bond | bond | 4-CH₂—N⁺(CH₃)₂-thien-3-yl, |
| | 3-yl | | | |
| 123 | 6-F-2H-chromen-3-yl | bond | bond | 4-CH₂—N⁺(CH₃)₂-cyclohexyl, |
| 124 | 5-F-2H-chromen-3-yl | bond | bond | 4-CH₂—N⁺(CH₃)₂-cyclohexyl, |
| 125 | 6-CF₃-2H-chromen- | bond | bond | 4-CH₂—N⁺(CH₃)₂-cyclohexyl, |
| | 3-yl | | | |
| 126 | 8-F-2H-chromen-3-yl | bond | bond | 4-CH₂—N⁺(CH₃)₂-cyclohexyl, |
| 127 | 7-CH₃-2H-chromen- | bond | bond | 4-CH₂—N⁺(CH₃)₂-cyclohexyl, |
| | 3-yl | | | |
| 128 | 7-OCH₃-2H- | bond | bond | 4-CH₂—N⁺(CH₃)₂-cyclohexyl, |
| | chromen-3-yl | | | |
| 129 | 6-OCH₃-2H- | bond | bond | 4-CH₂—N⁺(CH₃)₂-cyclohexyl, |
| | chromen-3-yl | | | |
| 130 | 6-CF₃-2H-chromen- | bond | bond | 4-CH₂—N⁺(CH₃)₂-thien-3-yl, |
| | 3-yl | | | |
| 131 | 4-F-2H-chromen-3-yl | bond | bond | 4-CH₂—N⁺(CH₃)₂-thien-3-yl, |
| 132 | 5-F-2H-chromen-3-yl | bond | bond | 4-CH₂—N⁺(CH₃)₂-thien-3-yl, |
| 133 | 4-CF₃-2H-chromen- | bond | bond | 4-CH₂—N⁺(CH₃)₂-cyclohexyl, |
| | 3-yl | | | |
| 134 | 8-CF₃-2H-chromen- | bond | bond | 4-CH₂—N⁺(CH₃)₂-cyclohexyl, |
| | 3-yl | | | |
| 135 | 3H- | bond | bond | 4-CH₂—N⁺(CH₃)₂-tetrahydro-pyran-4-yl, |
| | benzo[f]chromen-2- | | | |
| | yl | | | |
| 136 | 3H- | bond | bond | 4-CH₂-(1-CH₃-pyrrolidinium), |
| | benzo[f]chromen-2- | | | |
| | yl | | | |
| 137 | 3H- | bond | bond | 4-CH₂—N⁺(CH₃)₂-cyclohexyl, |
| | benzo[f]chromen-2- | | | |
| | yl | | | |
| 138 | 3H- | bond | bond | 4-CH₂—N⁺(CH₃)₂-tetrahydro-thiopyran-4- |
| | benzo[f]chromen-2- | | | yl, |
| | yl | | | |
| 139 | 3H- | bond | bond | 4-CH₂-(4-CH₃-morpholin-4-ium), |
| | benzo[f]chromen-2- | | | |
| | yl | | | |
| 140 | 3H- | bond | bond | 4-CH₂—N⁺(CH₃)₂-CH₂-tetrahydro-pyran- |
| | benzo[f]chromen-2- | | | 4-yl, |
| | yl | | | |
| 141 | 3H- | bond | —CH₂— | 4-CH₂—N⁺(CH₃)₂-tetrahydro-pyran-4-yl, |
| | benzo[f]chromen-2- | | | |
| | yl | | | |
| 142 | 3-Br-8,9-dihydro-7H- | bond | —CH₂— | 4-CH₂—N⁺(CH₃)₂-tetrahydro-pyran-4-yl, |
| | benzocyclohepten-6- | | | |
| | yl | | | |
| 143 | 3-Br-8,9-dihydro-7H- | bond | bond | 4-CH₂—N⁺(CH₃)₂-tetrahydro-pyran-4-yl, |
| | benzocyclohepten-6- | | | |
| | yl | | | |
| 144 | 3-Br-8,9-dihydro-7H- | bond | bond | 4-CH₂—N⁺(CH₃)₂-cyclohexyl, |
| | benzocyclohepten-6- | | | |
| | yl | | | |
| 145 | 8,9-dihydro-7H- | bond | bond | 4-CH₂-(1-CH₃-pyrrolidinium), |
| | benzocyclohepten-6- | | | |
| | yl | | | |
| 146 | 8,9-dihydro-7H- | bond | bond | 4-CH₂—N⁺(CH₃)₂-cyclohexyl, |
| | benzocyclohepten-6- | | | |
| | yl | | | |
| 147 | 8,9-dihydro-7H- | bond | bond | 4-CH₂—N⁺(CH₃)₂-tetrahydro-pyran-4-yl, |
| | benzocyclohepten-6- | | | |
| | yl | | | |
| 148 | 8,9-dihydro-7H- | bond | —CH₂— | 4-CH₂—N⁺(CH₃)₂-tetrahydro-pyran-4-yl, |
| | benzocyclohepten-6- | | | |
| | yl | | | |
| 149 | (2-CH₃-5-phenyl)- | bond | —CH₂— | 4-CH₂—N⁺(CH₃)₂-tetrahydro-pyran-4-yl, |
| | furan-3-yl | | | |
| 150 | [5-(4-Cl-phenyl)-2- | bond | —CH₂— | 4-CH₂—N⁺(CH₃)₂-tetrahydro-pyran-4-yl, |
| | CH₃]-furan-3-yl | | | |
| 151 | (2-CH₃-5-phenyl)- | bond | bond | 4-CH₂—N⁺(CH₃)₂-cyclohexyl, |
| | furan-3-yl | | | |
| 152 | benzofuran-2-yl | bond | bond | 4-CH₂—N⁺(CH₃)₂-tetrahydro-pyran-4-yl, |
| 153 | [5-(4-Cl-phenyl)-2- | bond | —CH₂— | 4-CH₂—N⁺(CH₃)₂-tetrahydro-pyran-4-yl, |
| | CF₃]-furan-3-yl | | | |
| 154 | [5-(4-Cl-phenyl)-2- | bond | —CH₂— | 4-CH₂—N⁺(CH₃)₂-cyclohexyl, |
| | CF₃]-furan-3-yl | | | |
| 155 | 5-Cl-benzofuran-2-yl | bond | bond | 4-CH₂—N⁺(CH₃)₂-tetrahydro-pyran-4-yl, |
| 156 | 5-Cl-benzofuran-2-yl | bond | bond | 4-CH₂—N⁺(CH₃)₂-cyclohexyl, |
| 157 | benzofuran-2-yl | bond | bond | 4-CH₂—N⁺(CH₃)₂-cyclohexyl, |
| 158 | 1-CH₃-1H-indol-2-yl | bond | bond | 4-CH₂—N⁺(CH₃)₂-tetrahydro-pyran-4-yl, |
| 159 | 5-Cl-1H-indol-2-yl | bond | bond | 4-CH₂—N⁺(CH₃)₂-tetrahydro-pyran-4-yl, |
| 160 | 5-Br-1H-indol-2-yl | bond | bond | 4-CH₂—N⁺(CH₃)₂-tetrahydro-pyran-4-yl, |
| 161 | 1-CH₃-1H-indol-3-yl | bond | bond | 4-CH₂—N⁺(CH₃)₂-tetrahydro-pyran-4-yl, |
| 162 | (1-CH₂-phenyl)-1H- | bond | bond | 4-CH₂—N⁺(CH₃)₂-tetrahydro-pyran-4-yl, |
| | indol-3-yl | | | |
| 163 | 1-CH₃-1H-indol-2-yl | bond | bond | 4-CH₂—N⁺(CH₃)₂-tetrahydro-pyran-4-yl, |
| 164 | 5-Cl-1H-indol-2-yl | bond | bond | 4-CH₂—N⁺(CH₃)₂-cyclohexyl, |
| 165 | 5-Cl-1H-indol-2-yl | bond | bond | 4-CH₂—N⁺(CH₃)₂-(2S)-CH₂-tetrahydro- |
| | | | | furan-2-yl, |
| 166 | 5-Cl-1H-indol-2-yl | bond | bond | 4-CH₂—N⁺(CH₃)₂-CH₂-bicyclo[2.2.1]hept- |
| | | | | 2-yl, |
| 167 | 7,8-Cl₂-2,3-dihydro- | bond | bond | 4-CH₂—N⁺(CH₃)₂-tetrahydro-pyran-4-yl, |
| | benzo[b]oxepin-4-yl | | | |
| 168 | 7,8-Cl₂-2,3-dihydro- | bond | bond | 4-CH₂—N⁺(CH₃)₂-cyclohexyl, |
| | benzo[b]oxepin-4-yl | | | |
| 169 | 7,8-Cl₂-2,3-dihydro- | bond | bond | 4-CH₂—N⁺(CH₃)₂-bicyclo[2.2.1]hept-2-yl, |
| | benzo[b]oxepin-4-yl | | | |
| 170 | 7,8-Cl₂-2,3-dihydro- | bond | —CH₂— | 4-CH₂—N⁺(CH₃)₂-tetrahydro-pyran-4-yl, |
| | benzo[b]oxepin-4-yl | | | |
| 171 | 7,8-Cl₂-2,3-dihydro- | bond | bond | 4-CH₂—N⁺(CH₃)₂-thien-3-yl, |
| | benzo[b]oxepin-4-yl | | | |
| 172 | 5-Br-pyridin-3-yl | bond | bond | 4-CH₂—N⁺(CH₃)₂-tetrahydro-pyran-4-yl, |
| 173 | 2-Cl-pyridin-4-yl | bond | bond | 4-CH₂—N⁺(CH₃)₂-tetrahydro-pyran-4-yl, |
| 174 | 3-Cl-benzo[b]thien- | bond | bond | 4-CH₂—N⁺(CH₃)₂-tetrahydro-pyran-4-yl, |
| | 2-yl | | | |
| 175 | 2,5-Cl₂-thien-3-yl | bond | bond | 4-CH₂—N⁺(CH₃)₂-tetrahydro-pyran-4-yl, |
| 176 | benzo[b]thien-2-yl | bond | bond | 4-CH₂—N⁺(CH₃)₂-tetrahydro-pyran-4-yl, |
| 177 | benzo[b]thien-2-yl | bond | bond | 4-CH₂—N⁺(CH₃)₂-cyclohexyl, |
| 178 | 3-Cl-benzo[b]thien- | bond | bond | 4-CH₂—N⁺(CH₃)₂-cyclohexyl, |
| | 2-yl | | | |

Phenylamino substituted quaternary salts of chemical formula, Formula A is the preferred pheynylamino substituted quaternary salt.

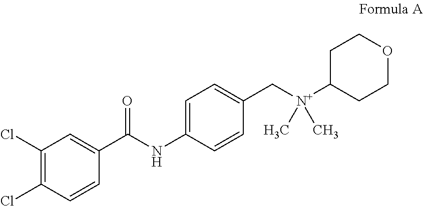

As used herein, the term “ionic ophthalmic devices” refers to ophthalmic devices made from a formulation that has a permanent charge. Examples of such ionic ophthalmic devices are made from the following USAN formulations which include but are not limited to etafilcon A, bufilcon A, deltafiln A droxifilcon A phemfilcon A ocufilcon A balafilcon A bufilcon A perifilcon A ocufilcon B, ocufilcon C ocufilcon D ocufilcon E, metafilcon A, metafilcon B, vifilcon A focofilcon A and tetrafilcon B. The preferred ionic ophthalmic devices are selected from the group consisting of etafilcon A, bufilcon A, deltafiln A droxifilcon A phemfilcon A ocufilcon A balafilcon A bufilcon A perifilcon A ocufilcon B, and ionic silicone hydrogels, prepared as disclosed by U.S. Pat. App. Pub. No. US 2010/0249356, which is hereby incorporated by reference in its entirety. The most preferred ionic ophthalmic devices are etafilcon A, and example 9 of U.S. Pat. App. Pub. No. US 2010/0249356.

The term “effective amount” refers to the weight of phenylamino substituted quaternary salts contained in an ionic ophthalmic device prior to its use by a patient wherein such effective amount alleviates the symptoms of CCR2 mediated inflammatory responses. The effective amount may vary depending upon the efficacy of a particular phenylamino substituted quaternary salts. For example, if the phenylamino substituted quaternary salt is Formula A, the weight percentage of salt, based on the weight of a hydrated lens is about 1% to about 2%. For example if the weight of a hydrated ophthalmic device is about 40 mg, the weight of phenylamino substituted quaternary salt incorporated into that device is about 0.763 mg to about 0.675 mg.

As used herein, “ophthalmic device” refers to an object that resides in or on the eye. These devices can provide optical correction or may be cosmetic. Ophthalmic devices include but are not limited to soft contact lenses, intraocular lenses, overlay lenses, ocular inserts, punctual plugs, and optical inserts. The preferred ophthalmic devices of the invention are soft contact lenses made from ionic formulations as described above.

Further the invention includes a method of alleviating the symptoms of CCR2 mediated inflammatory conditions comprising administering to a patient an ionic ophthalmic device comprising about an effective amount of an phenylamino substituted quaternary salt. The terms phenylamino substituted quaternary salts, ionic ophthalmic device, effective amount and pheynylamino substituted quaternary salt all have their aforementioned meanings and preferred ranges. As used herein, the term “administering” means placing the ophthalmic device of the invention onto the surface of the eye, or in the eye, of a patient. If the device is in contact with the anterior surface of the patient's eye, such as a soft contact lens, it is preferred that the ophthalmic device remain in contact with that surface for between about 5 minutes, and about 24 hours from insertion of the ophthalmic device into the eye of a user, more preferably between about 5 minutes and about 16 hours, more preferably between about 5 minutes and about 12 hours, most preferably between about 5 minutes and greater than about 12 hours. If the ophthalmic device is contained within the eye or on the ocular adnexa, such as a punctual plug or an ocular insert, it is preferred that the device remain in contact with the eye for at least 24 hours.

Still further the invention includes a method of making an ionic ophthalmic device comprising about an effective amount of a phenylamino substituted quaternary salt comprising the step of treating an ionic ophthalmic device with a solution comprising said phenylamino substituted quaternary salt, wherein the amount of said pheynylamino substituted quaternary salt in said solution exceeds the effective amount. It is preferred that the effective amount is exceeded by between about 30% and about 100%, in a volume of solution that is between about 500 μL and about 5000 μL preferably between about 40% and about 50%, in a volume of solution that is between about 500 μL and about 3000 μL most preferably about 50% in a volume of solution that is about 1000 μL.

As used herein treating means physical methods of contacting the solution containing an phenylamino substituted quaternary salt and the ophthalmic device. Preferably treating refers to physical methods of contacting the phenylamino substituted quaternary salt with the ionic ophthalmic devices prior to selling or otherwise delivering the ionic ophthalmic devices to a patient. The ionic ophthalmic devices may be treated with the phenylamino substituted quaternary salt anytime after they are polymerized. Polymerization refers to the process in which components of an ionic ophthalmic device including but not limited to monomers, pre-polymers, diluents, catalysts, initiators, tints, UV blockers, antibacterial agents, polymerization inhibitors, and the like are reacted by thermal, chemical, and light initiated curing techniques to produce a formed polymer. The preferred methods of polymerization are the light initiated techniques disclosed in U.S. Pat. No. 6,822,016 which is hereby incorporated by reference in its entirety. It is preferred that the polymerized ophthalmic devices be treated with phenylamino substituted quaternary salt at temperature of greater than about 50° C. For example in some processes to manufacture contact lenses, an un-polymerized, or partially polymerized formulation is placed between two mold halves, spincasted, or static casted and polymerized. See, U.S. Pat. Nos. 4,495,313; 4,680,336; 4,889,664, 3,408,429; 3,660,545; 4,113,224; and 4,197,266, all of which are incorporated by reference in their entirety. In the case of hydrogels, the ionic ophthalmic device formulation is a hardened disc that is subjected to a number of different processing steps including treating the polymerized ionic ophthalmic device with liquids (such as water, inorganic salts, or organic solutions) to swell, or otherwise equilibrate this polymerized ionic ophthalmic device prior to enclosing the polymerized ionic ophthalmic device in its final packaging. Polymerized ionic ophthalmic devices that have not been swelled or otherwise equilibrated are known as un-hydrated polymerized ionic ophthalmic devices. The addition of the phenylamino substituted quaternary salt to any of the liquids of this “swelling or “equilibrating” step at room temperature or below is considered “treating” the lenses with phenylamino substituted quaternary salt as contemplated by this invention. In addition, the polymerized un-hydrated ophthalmic devices may be heated above room temperature with the phenylamino substituted quaternary salt during swelling or equilibrating steps. The preferred temperature range is from about 50° C. for about 15 minutes to about sterilization conditions as described below, more preferably from about 50° C. to about 85° C. for about 5 minutes.

Examples of blister packages and sterilization techniques are disclosed in the following references which are hereby incorporated by reference in their entirety, U.S. Pat. Nos. D435,966; 4,691,820; 5,467,868; 5,704,468; 5,823,327; 6,050,398, 5,696,686; 6,018,931; 5,577,367; and 5,488,815. This portion of the manufacturing process presents another method of treating the ionic ophthalmic devices with phenylamino substituted quaternary salts, namely adding phenylamino substituted quaternary salts to a solution prior to sealing the package, and subsequently sterilizing the package. This is the preferred method of treating ophthalmic devices with phenylamino substituted quaternary salts.

Sterilization can take place at different temperatures and periods of time. The preferred sterilization conditions range from about 100° C. for about 8 hours to about 150° C. for about 0.5 minute. More preferred sterilization conditions range from about 115° C. for about 2.5 hours to about 130° C. for about 5.0 minutes. The most preferred sterilization conditions are about 124° C. for about 18 minutes.

The “solutions” that are used in methods of this invention may be water-based solutions. Typical solutions include, without limitation, saline solutions, other buffered solutions, and deionized water. The preferred aqueous solution is deioinized water or saline solution containing salts including, without limitation, sodium chloride, sodium borate, sodium phosphate, sodium hydrogenphosphate, sodium dihydrogenphosphate, or the corresponding potassium salts of the same. These ingredients are generally combined to form buffered solutions that include an acid and its conjugate base, so that addition of acids and bases cause only a relatively small change in pH. The buffered solutions may additionally include 2-(N-morpholino)ethanesulfonic acid (MES), sodium hydroxide, 2,2-bis(hydroxymethyl)-2,2′,2″-nitrilotriethanol, n-tris(hydroxymethyl)methyl-2-aminoethanesulfonic acid, citric acid, sodium citrate, sodium carbonate, sodium bicarbonate, acetic acid, sodium acetate, ethylenediamine tetraacetic acid and the like and combinations thereof. Preferably, the solution is a borate buffered or phosphate buffered saline solution or deionized water. The particularly preferred solution contains about 500 ppm to about 18,500 ppm sodium borate, most particularly preferred about 1000 ppm of sodium borate.

In order to illustrate the invention the following examples are included. These examples do not limit the invention. They are meant only to suggest a method of practicing the invention. Those knowledgeable in contact lenses as well as other specialties may find other methods of practicing the invention. However, those methods are deemed to be within the scope of this invention.

### EXAMPLES

### Example 1

### Preparation of Ophthalmic Devices Containing Formula A

The phenylamino substituted quaternary salt of Formula A was dissolved in 1-Day Acuvue packaging solution at a concentration of 0.5 mg/mL. The pH of the solution was adjusted to ca. 6.5.

1-Day Acuvue® Brand Contact Lenses (etafilcon A, an ionic contact lens) were removed from their packages and repackaged in glass vials containing 3.0 mL of the 0.5 g/mL Formula A solution described above. The vials were sealed with a Teflon coated stopper and heated for 18 minutes at 124° C.

After sterilization the packaging solution was evaluated to determine how much of the compound of Formula A was absorbed by the lens. The average amount of compound absorbed was 0.763 mg.

### Example 2

### Release of Formula A

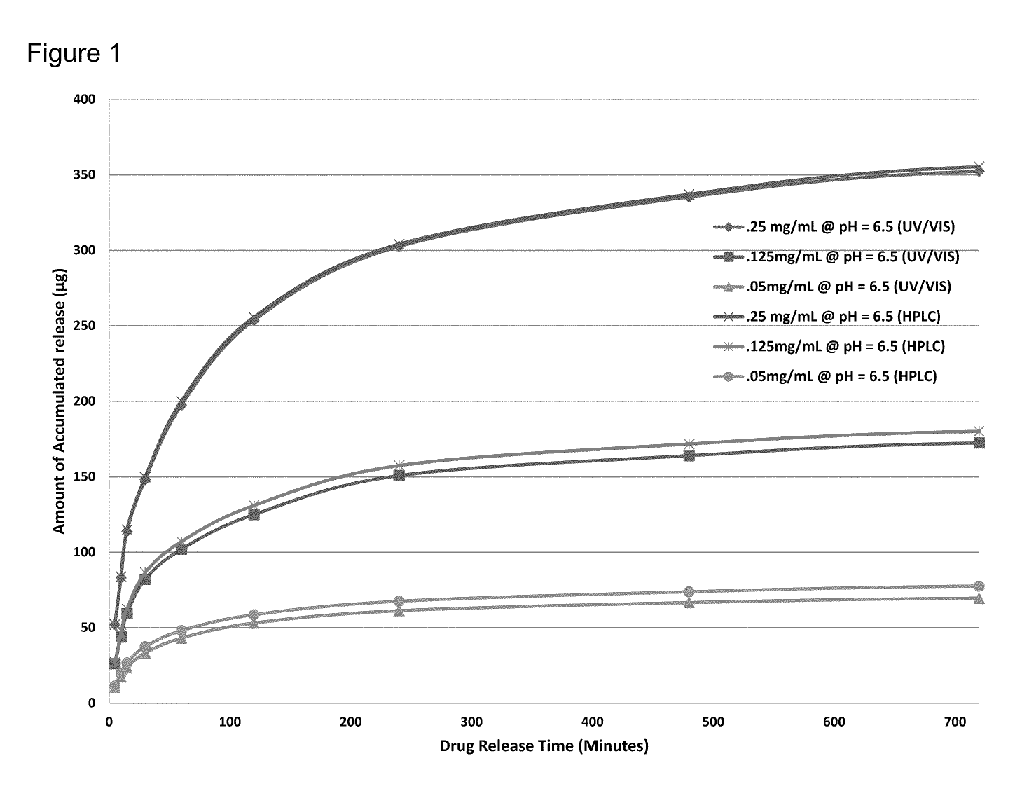

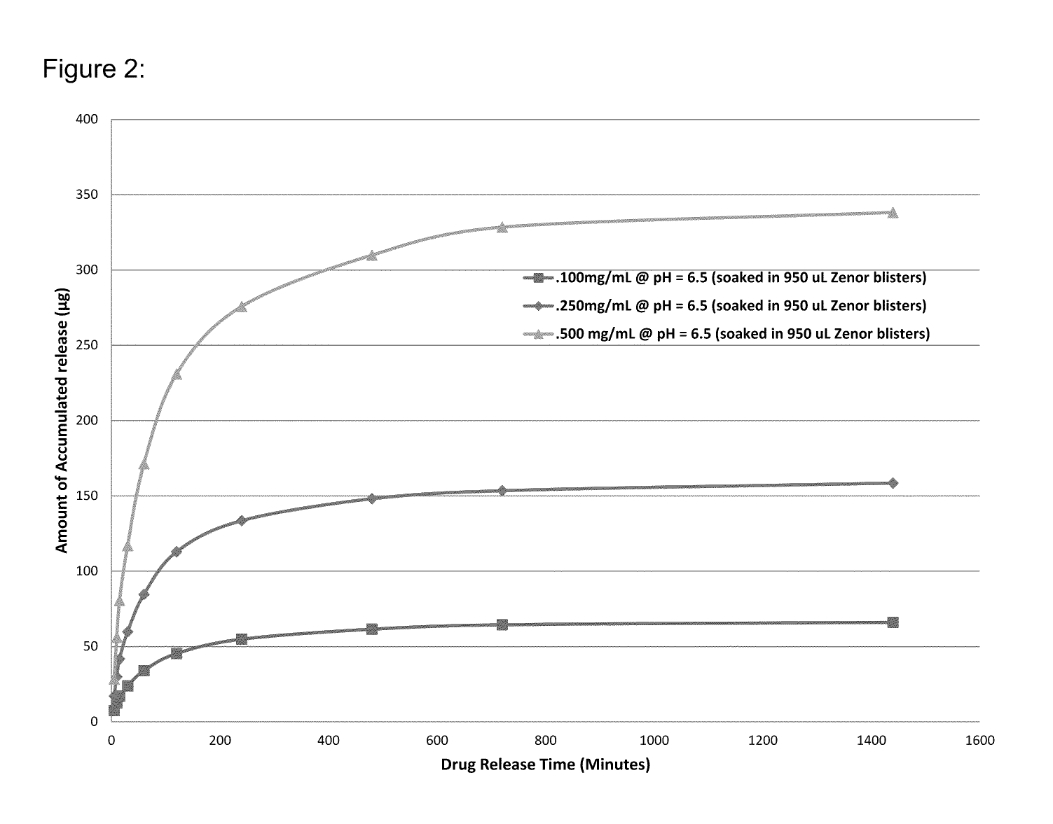

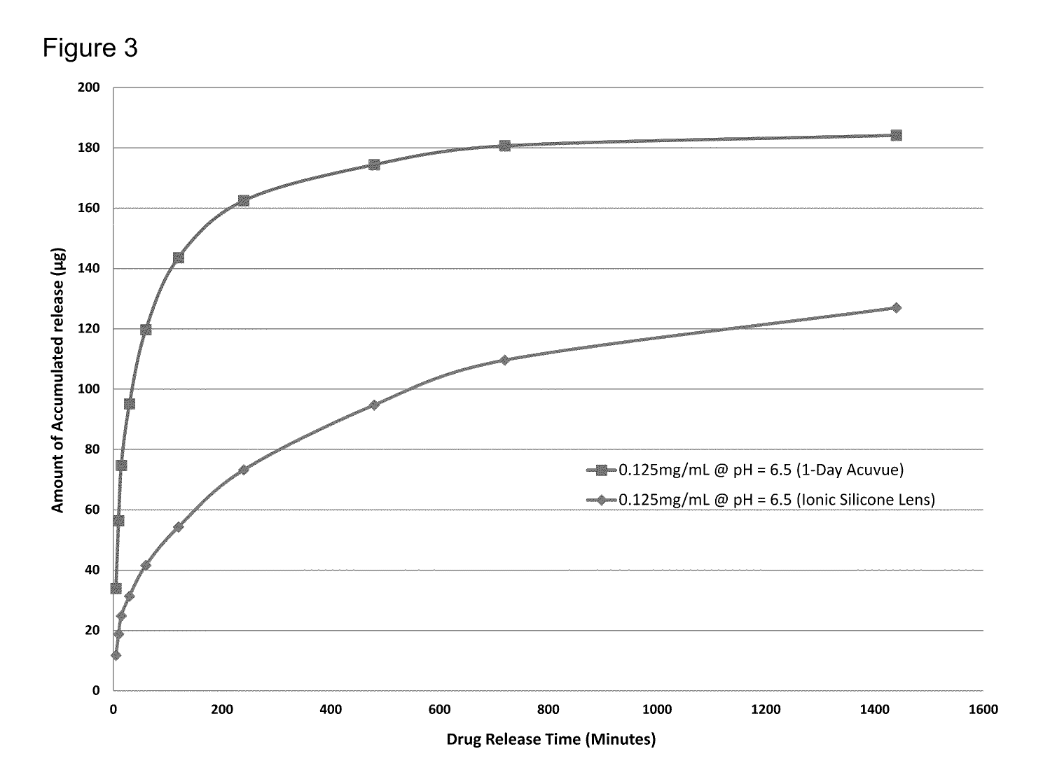

Lenses were prepared as in Example 1, except that three different concentrations of compound of Formula A were used, 0.05, 0.125, and 0.25 mg/mL respectively. Phosphate buffered saline, pH 7.4 (mL) was dispensed into a 20 mL glass scintillation vial. The sterilized lens was collected using cue-tip cotton swab, being careful to remove excess drug solution form the lens. Each lens was placed into the scintillation vial containing the PBS, sealed with a screw cap and placed in a shaking incubator at 37° C./50 rpm. At predetermined intervals of 5, 10, 15, 30, 60, 120, 240, 480, 720, and 1440, the lenses were removed and placed in fresh PBS. The PBS solutions were set aside for drug content analysis by UV-Vis and HPLC. FIG. 1 illustrates the release profile of the Formula A from etafilcon A lenses.

### Example 2

1-Day Acuvue® Brand Contact Lenses (etafilcon A, an ionic contact lens) were removed from their packages and repackaged in Zenor blister packages containing 950 μL of phosphate buffered saline, pH 7.4 containing the 0.1, 0.25, and 0.5 mg/mL of Formula A solutions respectively. The blister packages were sealed and sterilized, as described above. After sterilization the lenses were evaluated by HPLC to determine how much salt of Formula A was released over time. The results are presented in FIG. 2

### Example 3

The phenylamino substituted quaternary salt of Formula A was dissolved in 1-Day Acuvue packaging solution at a concentration of 0.125 mg/mL. The pH of the solution was adjusted to ca. 6.5.

Ionic silicone hydrogel lenses were prepared as disclosed in Example 9 of U.S. Pat. App. Pub. No. US 2010/0249356 (“Ionic Silicone Lens”). The Ionic Silicon Lenses were packaged in glass vials containing 3.0 mL of the 0.125 g/mL Formula A solution described above. The vials were sealed with a Teflon coated stopper and heated for 18 minutes at 124° C.

After sterilization the lenses were evaluated by HPLC to determine how much salt of Formula A was released over time. The results are presented in FIG. 3, with the release profile of etafilcon A lenses as prepared by the method of Example 1.

What is claimed is:

1. An ionic ophthalmic device comprising an effective amount of a phenylamino substituted quaternary salt

2. The ionic ophthalmic device of claim 1 wherein the pheynylamino substituted quaternary salt from the group consisting of compounds of 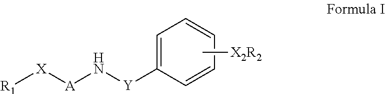
and pharmaceutically acceptable forms thereof, wherein
A is carbonyl, thiocarbonyl or sulfonyl;
X is a bond or —CH═CH—;
R₁ is selected from
aryl optionally substituted by one or more lower alkyl, —(CH₂)ₙ—CF₃, lower alkoxy, alkoxycarbonyl, cyano, halogen or phenyl optionally substituted by lower alkyl, —(CH₂)ₙ—CF₃, lower alkoxy, alkoxycarbonyl, cyano or halogen;
C₅-C₁₅ cycloalkyl optionally substituted by one or more lower alkyl, —(CH₂)ₙ—CF₃, lower alkoxy, aryl, halogen-substituted aryl, alkoxycarbonyl, cyano or halogen; or,
heterocyclyl optionally substituted by one or more lower alkyl, —(CH₂)ₙ—CF₃, lower alkoxy, aryl, aryl-lower alkyl, halogen-substituted aryl, alkoxycarbonyl, cyano or halogen;
n is 0, 1, 2, 3 or 4;
Y is a bond or —CH₂—;
X₂ is —(CH₂)ₘ— wherein m is 1 or 2;
R₂ is —N⁺(R₄R₅)—ZR₃;
Z is —(CH₂)ₚ— wherein p is 0, 1 or 2;
R₃ is selected from
aryl optionally substituted with one or more lower alkyl, —(CH₂)ₙ—CF₃, lower alkoxy, aryl, halogen-substituted aryl, alkoxycarbonyl, cyano or halogen;
C₅-C₁₅ cycloalkyl optionally substituted with one or more lower alkyl, —(CH₂)ₙ—CF₃, lower alkoxy, aryl, halogen-substituted aryl, alkoxycarbonyl, cyano or halogen; or,
heterocyclyl optionally substituted with one or more lower alkyl, —(CH₂)ₙ—CF₃, lower alkoxy, aryl, halogen-substituted aryl, alkoxycarbonyl, cyano or halogen; wherein, when heterocyclyl is attached via a carbon atom ring member and a heteroatom ring member is adjacent to said carbon atom, then p is 1 or 2;
R₄ and R₅ are each individually lower alkyl or lower alkenyl;
alternatively, R₄ and R₅ combine with the nitrogen atom of Formula (I) to form a heterocyclyl ring of 5 to 9 total ring atoms optionally containing one of an oxygen or sulfur ring atom, wherein the heterocyclyl ring nitrogen atom is substituted with one of lower alkyl or lower alkenyl to form a quaternary salt, and wherein —ZR₃ is absent and the heterocyclyl ring is optionally substituted with aryl optionally substituted with one or more lower alkyl, —(CH₂)ₙ—CF₃, lower alkoxy, aryl, halogen-substituted aryl, alkoxycarbonyl, cyano or halogen.

3. The ionic ophthalmic device of claim 1 wherein the pheynylamino substituted quaternary salt is a compound of Formula A.

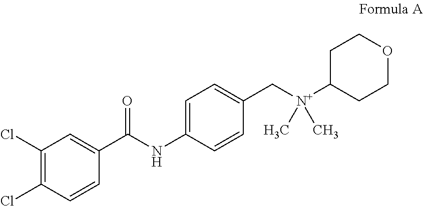

4. The ionic ophthalmic device of claim 1 wherein the ionic ophthalmic devices is a cured a formulation selected from the group consisting of etafilcon A, bufilcon A, deltafiln A droxifilcon A phemfilcon A ocufilcon A balafilcon A bufilcon A perifilcon A ocufilcon B, ocufilcon C ocufilcon D ocufilcon E, metafilcon A, metafilcon B, vifilcon A focofiln A and tetrafilcon B.

5. The ionic ophthalmic device of claim 1 wherein the ionic ophthalmic devices is a cured a formulation selected from the group consisting of etafilcon A, bufilcon A, deltafiln A droxifilcon A phemfilcon A ocufilcon A balafilcon A bufilcon A perifilcon A and ocufilcon B.

6. The ionic ophthalmic device of claim 1 wherein the ionic ophthalmic devices is a cured an etafilcon formulation.

7. The ionic ophthalmic device of claim 3 wherein the ionic ophthalmic device is etafilcon A.

8. The ionic ophthalmic device of claim 7 wherein the effective amount of the compound of Formula A is about 1% to about 2% by weight based on the weight of the hydrated ionic ophthalmic device.

9. A method of alleviating the symptoms of CCR2 mediated inflammatory conditions comprising administering to a patient an ionic ophthalmic device comprising about an effective amount of an phenylamino substituted quaternary salt.

10. A method of making an ionic ophthalmic device comprising about an effective amount of a phenylamino substituted quaternary salt comprising the step of treating an ionic ophthalmic device with a solution comprising said phenylamino substituted quaternary salt, wherein the amount of said pheynylamino substituted quaternary salt in said solution exceeds the effective amount.
